(19) United States
(12) Patent Application Publication
(10) Pub. No.: US 2024/0408291 A1
(43) Pub. Date: Dec. 12, 2024

(54) DEVICES, SYSTEMS AND METHODS FOR TREATING THE SKIN

(71) Applicant: HydraFacial LLC, Long Beach, CA (US)

(72) Inventors: Roger G. Ignon, Redondo Beach, CA (US); Ed F. Nicolas, Signal Hill, CA (US); Johann Van Bel, Huntington Beach, CA (US); David Hernandez, Long Beach, CA (US); Jesse Robbers, Garden Grove, CA (US); Michael Palmer, Irvine, CA (US); James Foster, Irvine, CA (US); Kevin Oberkramer, Irvine, CA (US); Kevin Christopher, Irvine, CA (US); Yale Shaw, San Francisco, CA (US); Elliot Ortiz, Pinole, CA (US)

(21) Appl. No.: 18/700,255

(22) PCT Filed: Oct. 7, 2022
(86) PCT No.: PCT/US2022/077811

§ 371 Date: Apr. 10, 2024

Related U.S. Application Data
(60) Provisional application No. 63/254,455, filed on Oct. 11, 2021.

(51) Int. Cl. A61M 1/00; A61B 17/32

(52) U.S. Cl. CPC A61M 1/774; A61B 17/32; A61M 1/74; A61B 2017/320004; A61M 2205/502; A61M 2205/60; A61M 2210/04

(57) ABSTRACT

According to some embodiments, a skin treatment system comprises a manifold assembly comprising at least one fluid connector to secure at least one treatment fluid container, a primary handpiece configured to hydraulically couple to the manifold assembly, wherein the primary handpiece is hydraulically coupled to the manifold assembly using at least one fluid conduit, and a flow control device positioned along the at least one fluid conduit, the flow control device configured to switch flow of treatment fluid from the at least one treatment fluid between the primary handpiece and a port, wherein the port is configured to be coupled to a secondary handpiece, wherein a vacuum source is configured to operatively couple to the system and selectively create suction along the primary handpiece.

### CROSS REFERENCE TO RELATED APPLICATIONS

This application claims the priority benefit of U.S. Provisional Patent Application No. 63/254,455, filed Oct. 11, 2021, the contents of which are incorporated by reference herein in their entirety.

### FIELD

This application relates generally to skin treatment, and more specifically, to apparatuses, systems and methods for treating a person's skin.

### DESCRIPTION OF THE RELATED ART

Abrasion of the outer layer or epidermis of the skin is desirable to smooth or blend scars, blemishes, or other skin conditions that may be caused by, for example, acne, sun exposure, and aging. Standard techniques used to abrade the skin have generally been separated into two fields referred to as dermabrasion and microdermabrasion. Both techniques remove portions of the epidermis called the stratum corneum, which the body interprets as a mild injury. The body then replaces the lost skin cells, resulting in a new outer layer of skin. Additionally, despite the mild edema and erythema associated with the procedures, the skin looks and feels smoother because of the new outer layer of skin.

### SUMMARY

According to some embodiments, a skin treatment system comprises a manifold assembly comprising at least one fluid connector to secure at least one treatment fluid container, a primary handpiece configured to hydraulically couple to the manifold assembly, wherein the primary handpiece is hydraulically coupled to the manifold assembly using at least one fluid conduit, and a flow control device positioned along the at least one fluid conduit, the flow control device configured to switch flow of treatment fluid from the at least one treatment fluid between the primary handpiece and a port, wherein the port is configured to be coupled to a secondary handpiece, wherein a vacuum source is configured to operatively couple to the system and selectively create suction along the primary handpiece.

According to some embodiments, the vacuum source is configured to create suction along the port. In some embodiments, the port is not configured to be in fluid communication with the vacuum source.

According to some embodiments, the flow control device comprises a valve. In some arrangements, the valve comprises a three-way valve. In some embodiments, the flow control device is manually controlled. In some embodiments, the flow control device is automatically controlled. In some embodiments, the flow control device is automatically controlled based on a skin treatment protocol being performed.

According to some embodiments, the flow control device is automatically controlled based on one or more inputs from a user. In some embodiments, the one or more inputs are provided using a user input device. In some embodiments, the user input device comprises a touchscreen, a keyboard, a button, a switch or another device.

According to some embodiments, the flow control device is automatically controlled by, at least in part, a control module of the system. In some embodiments, the manifold assembly is configured to receive at least two treatment fluid containers.

According to some embodiments, the manifold assembly comprises at least one automatic tag reader, wherein the tag reader is configured to obtain information from a tag positioned on the at least one treatment fluid container when the at least one treatment fluid container is secured to the manifold assembly. In some configurations, the at least one automatic tag reader comprises a RFID reader.

According to some embodiments, the system is configured to receive at least one command from a user using a gesture control system. In some embodiments, the gesture control system comprises at least two gesture sensors. In one embodiment, the gesture control system comprises at least four gesture sensors.

According to some embodiments, the system further comprises at least one flow meter configured to measure a flow rate of fluid passing through the at least one fluid conduit. In some embodiments, treatment fluid is delivered from the manifold assembly to the at least one fluid conduit using a pulsed pattern. In some embodiments, the pulsed pattern is created by actuating an air release valve in fluid communication with the at least one fluid conduit.

According to some embodiments, a method of treating skin using a skin treatment system comprises transferring at least one treatment fluid from a tower assembly to a primary handpiece, wherein the tower assembly comprises a manifold, wherein the manifold comprises at least one fluid connector to secure at least one treatment fluid container, and wherein the primary handpiece is configured to hydraulically couple to manifold using at least one fluid conduit. The method further includes switching flow of the at least one treatment fluid along the at least one fluid conduit using a flow control device, wherein the flow control device is configured to switch flow of the at least one treatment fluid between the primary handpiece and a port, wherein the port is configured to be coupled to a secondary handpiece, wherein a vacuum source is configured to generate a suction force along the primary handpiece.

According to some embodiments, a skin treatment system comprises a tower assembly (e.g., console) including a manifold assembly, the manifold assembly comprising at least one fluid connector to secure at least one treatment fluid container, a primary handpiece assembly configured to hydraulically couple to the tower assembly, wherein the primary handpiece assembly is hydraulically coupled to the tower assembly using at least one fluid conduit, a valve positioned along the at least one fluid conduit, the valve configured to switch flow of treatment fluid from the at least one treatment fluid between the primary handpiece assembly and a port, wherein the port is configured to be coupled to a secondary handpiece assembly, and a vacuum source (e.g., vacuum pump) configured to generate a suction force along the primary handpiece assembly.

According to some embodiments, the secondary handpiece assembly is not configured to be in fluid communication with the vacuum source.

According to some embodiments, the valve comprises a three-way valve. In some embodiments, the valve is manually controlled.

According to some embodiments, the valve is automatically controlled. In some arrangements, the valve is automatically controlled based on a skin treatment protocol being performed. In some embodiments, the valve is automatically controlled based on one or more inputs from a user. In some embodiments, the one or more inputs are provided using a user input device (e.g., a touchscreen). In some embodiments, the valve is automatically controlled by, at least in part, a control module of the system.

According to some embodiments, the manifold assembly is configured to receive at least two treatment fluid containers (e.g., two, three, four, etc.).

According to some embodiments, the manifold assembly comprises at least one automatic tag reader, wherein the tag reader is configured to obtain information from a tag positioned on the at least one treatment fluid container when the at least one treatment fluid container is secured to the manifold assembly. In some embodiments, the at least one automatic tag reader comprises a RFID reader.

According to some embodiments, the system is configured to receive at least one command from a user using gesture control technology.

According to some embodiments, the system further comprises at least one flow meter configured to measure a flow rate of fluid passing through the at least one fluid conduit.

According to some embodiments, treatment fluid is delivered from the manifold assembly to the at least one fluid conduit using a pulsed pattern. In some embodiments, the pulsed pattern is created by actuating an air release valve in fluid communication with the at least one fluid conduit.

According to some embodiments, a method of treating skin using a skin treatment system comprises transferring at least one treatment fluid from a tower assembly to a primary handpiece assembly, wherein the tower assembly comprises a manifold assembly, wherein the manifold assembly comprises at least one fluid connector to secure at least one treatment fluid container, and wherein the primary handpiece assembly is configured to hydraulically couple to the tower assembly, wherein the primary handpiece assembly is hydraulically coupled to the tower assembly using at least one fluid conduit. The method further comprises switching flow of the at least one treatment fluid along the at least one fluid conduit using a valve, wherein the valve is configured to switch flow of the at least one treatment fluid between the primary handpiece assembly and a port, wherein the port is configured to be coupled to a secondary handpiece assembly, wherein a vacuum source is configured to generate a suction force along the primary handpiece assembly.

According to some embodiments, the secondary handpiece assembly is not configured to be in fluid communication with the vacuum source.

According to some embodiments, the valve comprises a three-way valve. In some embodiments, the valve is manually controlled.

According to some embodiments, the valve is automatically controlled. In some arrangements, the valve is automatically controlled based on a skin treatment protocol being performed. In some embodiments, the valve is automatically controlled based on one or more inputs from a user. In some embodiments, the one or more inputs are provided using a user input device (e.g., a touchscreen). In some embodiments, the valve is automatically controlled by, at least in part, a control module of the system.

According to some embodiments, the manifold assembly is configured to receive at least two treatment fluid containers (e.g., two, three, four, etc.).

According to some embodiments, the manifold assembly comprises at least one automatic tag reader, wherein the tag reader is configured to obtain information from a tag positioned on the at least one treatment fluid container when the at least one treatment fluid container is secured to the manifold assembly. In some embodiments, the at least one automatic tag reader comprises a RFID reader.

According to some embodiments, the system is configured to receive at least one command from a user using gesture control technology.

According to some embodiments, the method further comprises measuring a flow rate of fluid passing through the at least one fluid conduit using at least one flow meter.

According to some embodiments, treatment fluid is delivered from the manifold assembly to the at least one fluid conduit using a pulsed pattern. In some embodiments, the pulsed pattern is created by actuating an air release valve in fluid communication with the at least one fluid conduit.

According to some embodiments, a method of treating skin using a skin treatment system comprises transferring a first treatment fluid from a first container (e.g., bottle) to a handpiece assembly of the skin treatment system, transferring a second treatment fluid from a second container (e.g., bottle) to the handpiece assembly, wherein skin treatment system comprises a tower assembly having a manifold assembly, the manifold assembly being configured to receive the first container and the second container, wherein the manifold assembly comprises a main fluid conduit, the main fluid conduit combining fluid being transferred from the first container and/or the second container, wherein the handpiece assembly is configured to hydraulically couple to the manifold assembly of the tower assembly via the main fluid conduit, wherein the manifold assembly comprises a first branch conduit that places the first container in fluid communication with the main fluid conduit, and wherein the manifold assembly further comprises a second branch conduit that places the second container in fluid communication with the main fluid conduit. The method further comprises regulating a flow of treatment fluids through the first branch conduit and the second branch conduit to transfer a desired amount of the first treatment fluid and the second treatment fluid to the main fluid conduit and the handpiece assembly.

According to some embodiments, regulating the flow of treatment fluids comprises modulating at least one valve fluidly coupled to the first branch conduit and/or the second branch conduit. In some embodiments, the at least one valve is fluidly coupled to each of the first branch conduit and the second branch conduit. In some embodiments, the at least one valve is controlled automatically. In other embodiments, the at least one valve is controlled manually.

According to some embodiments, each of the first branch conduit and the second branch conduit comprises or is in fluid communication with a flowrate meter, the flowrate meter being configured to determine a flowrate of treatment fluid passing through the first or second branch conduit. In some embodiments, the main fluid conduit comprises a combined flowrate meter, the combined flowrate meter being configured to determine a total of flowrate of treatment fluids being delivered through the main fluid conduit to the handpiece assembly.

According to some embodiments, a processor of the skin treatment system (or a processor operatively coupled to the skin treatment system) is configured to regulate a flowrate of treatment fluids passing through the first branch conduit, the second branch conduit and the main fluid conduit.

According to some embodiments, a tip configured to be secured to a distal end of a handpiece of a skin treatment system comprises a main body portion having a distal end and a proximal end, the proximal end being configured to secure to the handpiece, a peripheral lip along the distal end, the peripheral lip defining an interior area, a rollerball positioned within the interior area, the rollerball secured to the tip and configured to rotate when the rollerball is moved relative to a skin surface, and at least one vacuum port terminating within the interior area and adjacent the rollerball, the at least one vacuum being configured to generate a suction or vacuum along the tip to remove spent fluids and other debris, wherein the at least one vacuum port is in fluid communication with a vacuum passage or conduit of the tip, the vacuum passage or conduit configured to hydraulically couple to a vacuum or suction source when the tip is secured to the handpiece.

According to some embodiments, the rollerball extends past the peripheral lip or is proud relative to the peripheral lip. In some embodiments, the interior area comprises an oval or egg shape. In some embodiments, the rollerball is located along a larger diameter portion of the interior area and the at least one vacuum port is located along a smaller diameter portion of the interior area. In some embodiments, the interior area comprises a circular shape.

According to some embodiments, a plane defined by a distal end of the peripheral lip is angled relative to a longitudinal axis of the tip and an axis orthogonal to the longitudinal axis.

According to some embodiments, the peripheral lip is configured to abrade skin tissue when the tip contacts skin tissue and is moved relative to skin tissue. In some embodiments, the peripheral lip comprises a sharp edge and/or an abrasive structure.

### BRIEF DESCRIPTION OF THE DRAWINGS

These and other features, aspects and advantages of the present application are described with reference to drawings of certain embodiments, which are intended to illustrate, but not to limit, the present inventions. It is to be understood that these drawings are for the purpose of illustrating the various concepts disclosed herein and may not be to scale.

FIG. 1 illustrates a perspective view of a tower assembly and other components of a skin treatment system according to one embodiment;

FIG. 2A schematically illustrates a flow diagram for a skin treatment system according to one embodiment;

FIG. 2B schematically illustrates a flow diagram for a skin treatment system according to another embodiment;

FIG. 3A schematically illustrates a flow diagram for a skin treatment system according to one embodiment;

FIG. 3B schematically illustrates a flow diagram for a skin treatment system according to another embodiment;

FIG. 3C schematically illustrates a flow diagram for a manifold region of a skin treatment system according to one embodiment;

FIG. 4A illustrates a perspective view of handpiece assembly configured for use in a skin treatment system, according to one embodiment;

FIG. 4B illustrates a side view of the handpiece assembly of FIG. 4A;

FIG. 5 illustrates a side view of a cartridge configured to be inserted within the handpiece assembly of FIG. 4A;

FIGS. 6A and 6B illustrate perspective views of different embodiments of tips configured to secure along a distal end of a handpiece assembly of a skin treatment system;

FIGS. 7A to 7C illustrate different views of one embodiment of a tip having a rollerball and being configured to secure along a distal end of a handpiece assembly;

FIGS. 7D to 7K illustrate different views of one embodiment of a tip identical or similar to the embodiment depicted in FIGS. 7A to 7C;

FIGS. 8A and 8B illustrate a manifold assembly of a skin treatment system and a bottle configured to secure thereto according to one embodiment;

FIGS. 9A and 9B illustrate vials and a corresponding tag reader portion of a tower assembly according to one embodiment; and

FIGS. 10A and 10B illustrate different views of a bottle configured to be positioned in a manifold assembly of a skin treatment system according to one embodiment;

### DETAILED DESCRIPTION

Although the various embodiments of a handpiece assembly have specific relevance to a skin treatment system, the features, advantages and other characteristics disclosed herein may have direct or indirect applicability in other applications, such as, for example, medical devices, mechanical devices and/or the like.

Several embodiments of the inventions disclosed herein are particularly advantageous because they include one, several or all of the following benefits: provide for enhanced delivery of treatment fluids to the skin of a subject; provide for delivery of fluids to the skin of a subject while reducing the likelihood of contamination; provide for enhanced collection of data regarding a skin treatment procedure; provide for enhanced treatment protocols based on data collection and processing; and provide for enhanced safety and other counterfeiting measures related to fluids delivered by skin treatment systems.

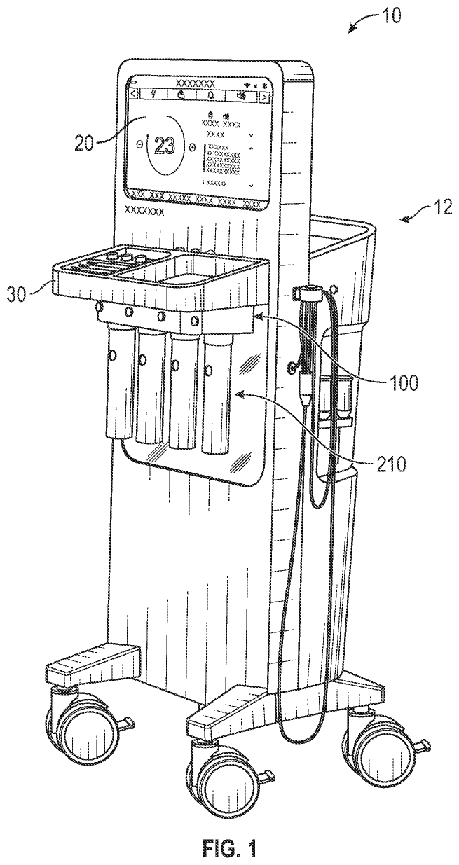

FIG. 1 illustrates one embodiment of a skin treatment system 10. As shown, the system 10 can include a tower assembly 12 which includes various components and features of the system. For example, in some embodiments, the tower assembly 12 comprises a manifold assembly 100 for receiving one or more (e.g., 1, 2, 3, 4, more than 4, etc.) bottles or other containers of treatment fluids to be used in a skin treatment procedure. The tower assembly 12 can further include one or more waste canisters or other containers that are configured to receive spent fluids, exfoliated or otherwise removed skin tissue and/or other waste products resulting from a skin treatment procedure. In some arrangements, one or more of the bottles, canisters or other containers that are designed and otherwise adapted to be secured to the tower assembly 12 can be replaceable, interchangeable and/or otherwise removable (e.g., for emptying, autoclaving or other types of cleaning, replacement, etc.).

The tower assembly 12 can further comprise (and/or can be configured to communicate or work with) one or more input and/or output devices (e.g., a touchscreen or other monitor 20, a keyboard, other controllers, etc.), an outer housing or other exterior structure, tubing, one or more trays or other storage components 30, casters or other wheels 18, interior components (e.g., processor, memory, power source, sensors, tubing, valves and/or other hydraulic components, electrical wiring and other electrical components, etc.) and/or the like.

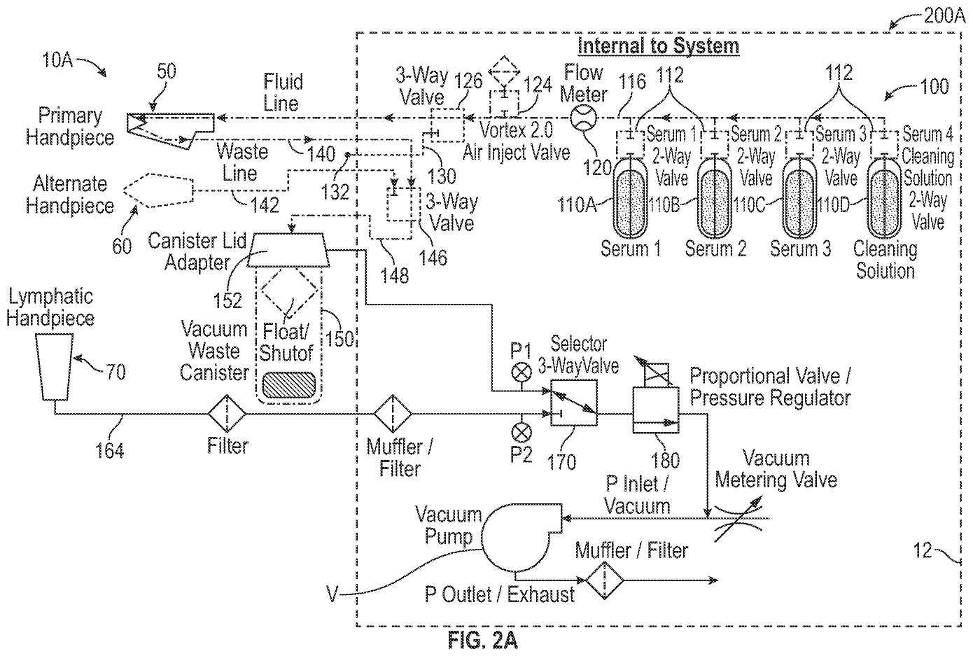

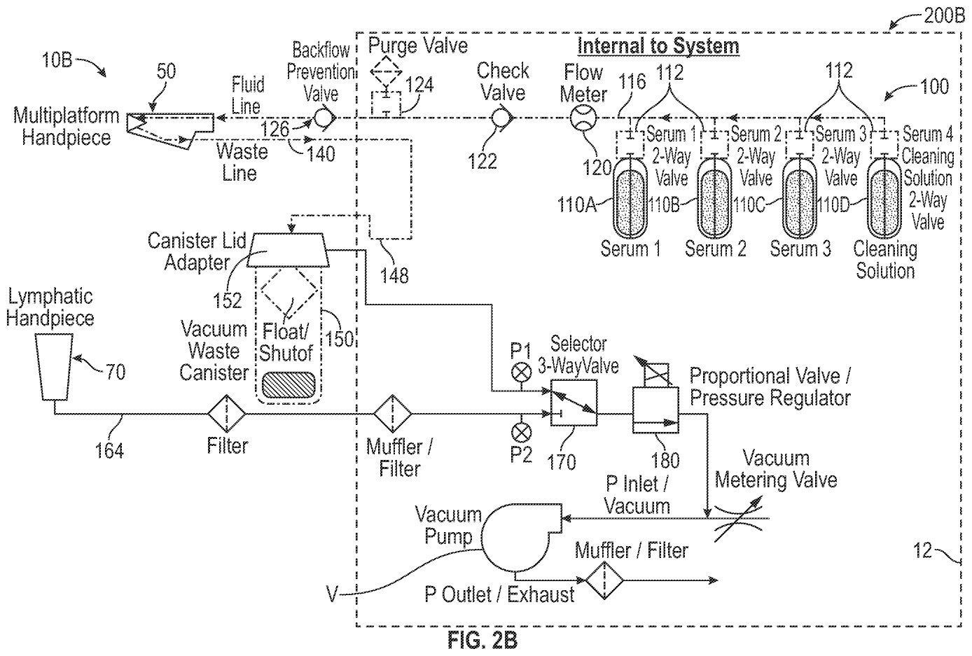

FIG. 2A schematically illustrates one embodiment of a flow diagram 200A that may be used in connection with a skin treatment system 10A. As shown, components of the system 10A can be included within and/or on console assembly 12. The system 10A can be configured to include and/or to be used with one or more handpiece assemblies (e.g., handpieces, other handheld devices, etc.) 50, 60, 70, as desired or required. The system 10A can include a suction or vacuum source V (e.g., vacuum pump). In some embodiments, the suction or vacuum source V in included with the system; however, in other arrangements, the system is configured to selectively couple to a suction or vacuum source V (e.g., a suction or vacuum source that is separate of the system).

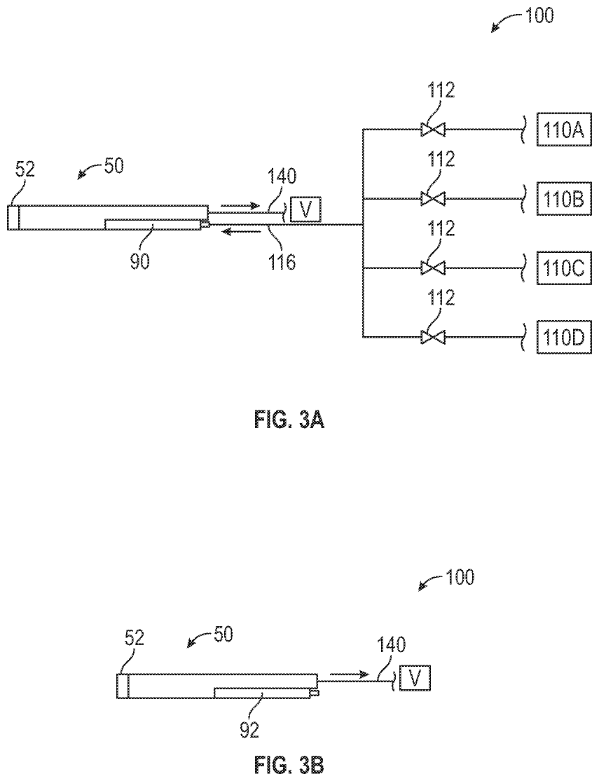

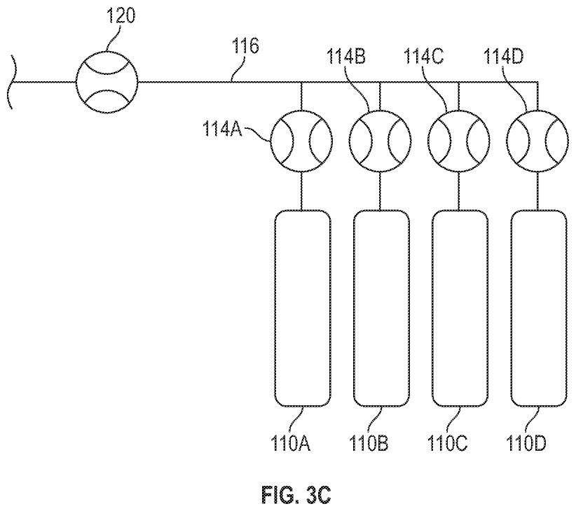

According to some embodiments, once activated, the vacuum source V can generate suction (e.g., negative pressure relative to atmospheric or ambient pressure) along the distal end (e.g., the tip) of a handpiece assembly. In some arrangements, as depicted in FIGS. 3A and 3B, such a suction or vacuum force along the tip or distal end of a handpiece can help draw one or more treatment fluids to the tip or distal end (e.g., via a manifold assembly 100, via a vial or cartridge positioned within the handpiece assembly, etc.). In some embodiments, the tip or distal end includes a peripheral lip or member that is configured to contact skin tissue. Such a lip or other peripheral member can form at least a partial seal with skin and help generate a vacuum or suction force within an interior area circumscribed by the lip or member. In turn, according to some arrangements, the vacuum or suction force along such an interior area along the tip or distal end can help draw fluids and/or other treatment materials from one or more sources to the tip or distal end (e.g., using the suction or vacuum alone without the need for positive pressure exerted on the fluids or other treatment materials being transferred to the tip or distal end).

According to certain embodiments, as schematically illustrated in FIGS. 3A and 3C, one or more bottles or other containers 110A, 110B, 110C, 110D can be placed in fluid communication with a manifold assembly 100 or other multi-branch assembly or system. In some arrangements, as illustrated in FIG. 3A, one or more valves or other flow control devices or components 112 can be positioned between each fluid source (e.g., a bottle) and a main delivery conduit or other fluid line 116 that places the manifold assembly 100 in fluid communication with a handpiece assembly 50. The valves or other flow control devices 112, which can include 2-way valves, can be configured to regulate the flow of treatment materials from one or more of the bottles or other containers 110A, 110B, 110C, 110D to the main delivery conduit 116, and thus, to the handpiece assembly 50.

With continued reference to FIG. 3A, the handpiece assembly 50 can be placed in fluid communication with a suction source (e.g., vacuum pump, not shown) using one or more vacuum or suction conduit or other fluid line 140. In the arrangements disclosed herein, the manifold assembly 100 comprises a total of four fluid branches. However, a system can comprise more or fewer fluid branches (e.g., 1, 2, 3, 4, 5, 6, 7, 8, more than 8, etc.), as desired or required by a particular application or use. Further, one or more of the bottles or other containers 110A, 110B, 110C, 110D that are positioned within the manifold assembly 100 can include a cleaning solution (e.g., for the periodic cleaning and/or flushing of the manifold assembly and/or other components with which a treatment fluid or other material may come in contact).

According to certain embodiments, one or more of the fluid conduits of the manifold system illustrated in FIG. 3A are configured to receive and transfer a serum, other treatment fluid and/or the like. Alternatively, however, one or more of the conduits can be configured to receive and transfer water (e.g., distilled, tap water, filtered, etc.), saline, other dilutants or dissolvents, other fluids and/or the like to the handpiece assembly 50. Such fluids can be adapted to, at least partially, contact, dissolve, dilute, liquefy, soften and/or otherwise mix with one or more solids, gels and/or other materials positioned within or on various surfaces or portions of the handpiece assembly 50 (e.g., tip). In some arrangements, this can provide a convenient method of delivering one or more materials at the skin-tip interface and/or any other location where such materials are desired or required.

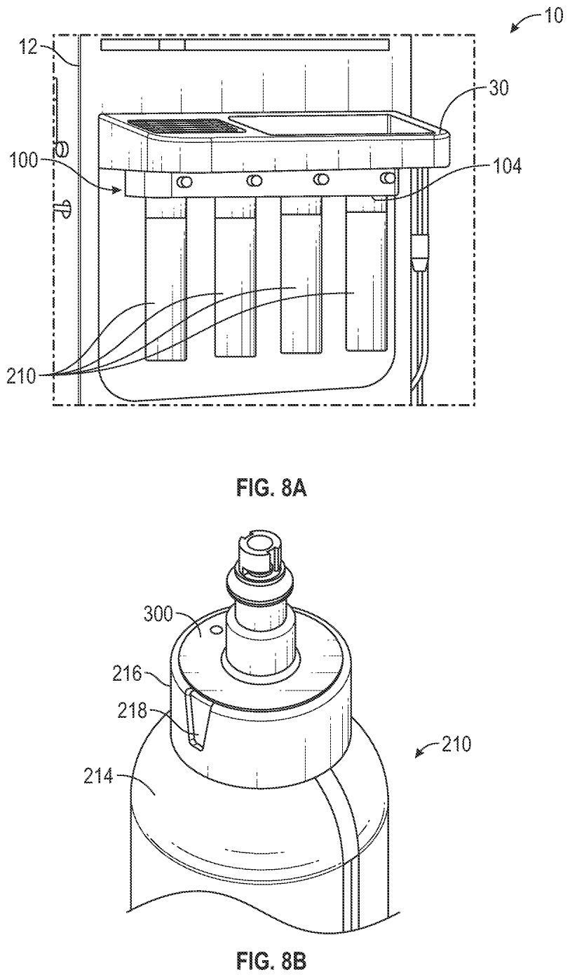

As discussed in additional detail in connection with FIG. 8 herein, a tower assembly 12 of a skin treatment system 10 can be provided with one or more identification components, tools and/or other features that advantageously provide data or other information to the user, manufacturer or supplier and/or any other party or entity associated with skin treatment procedures performed by a particular system. For instance, the system 10 can be configured to identify one or more RFID tags or other identifiers (e.g., other electromagnetic identifiers, graphic or other visual identifiers such as QR codes, barcodes, alphanumeric codes, etc.) secured to or otherwise associated with a serum or other treatment fluid container. The tags or other identifiers can help ensure that the correct serums and other treatment serums are being utilized for a specific skin procedure or other protocol.

With continued reference to the schematic of FIG. 2A, one or more flow meters 120 or other flowrate measuring (e.g., measuring, estimating, etc.) devices can be included in the fluid path placing the manifold assembly 100 in fluid communication with one or more handpiece assemblies 50, 60. Such a flow meter 120 can be used to measure or estimate the amount of fluid (e.g., treatment fluid, cleaning solution, etc.) that is being transferred from bottles or other containers coupled to the manifold assembly 100.

In some embodiments, the measured or estimated flow rate can be an actual flow rate quantity. However, in other embodiments, the flow rate can a relative flow rate (e.g., as a fraction or percentage) vis-à-vis one or more other flow rate measurements. For instance, with reference to the schematic illustrated in FIG. 3C and discussed in greater detail below, the relative flow rate of fluids and/or other materials posing through one or more flow meters 114A, 114B, 114C, 114D can be detected, measured or otherwise approximated. Such a relative flow can be a fraction or a percentage of flow for one of the flow meters, a main flow meter 120 and/or the like.

The measurement or approximation of flow rate can help confirm the proper function and/or use of the skin treatment system 10. For example, the flow meter 120 can help ensure that an accurate amount of treatment fluid is being delivered to a handpiece assembly. The use of flow meters 120 can help identify leaks, misuse and/or the like. In some embodiments, the flow meter 120 comprises an electromechanical flow meter. However, any type of flow meter 120 can be used, including, without limitation, a positive displacement flow meter, a velocity flow meter (e.g., turbine, paddle wheel, ultrasonic, electromagnetic, vortex-shedding, ultrasonic flow meters, etc.), mass flow meters or any other type of flow meter, as desired or required.

According to some embodiments of a skin treatment system, one or more valves or other flow control devices can be positioned adjacent (e.g., near, upstream, downstream relative to, etc.) one or more flow meters. The flow control device can be located away from the corresponding flow meter as long as the flow through the branch of conduit for which the flow meter determines or estimates flow rate is controlled, at least in part, by the flow control device.

In some arrangements, one or more aspects of such flow control devices can be modified to regulate the flow of fluid and/or other materials through one or more valves or other flow control devices. In some embodiments, such modification or regulation can be based, at least in part, on the measurements or estimations provided by one or more flow meter devices of the systems. For instance, a system can include a valve or other flow control device along one or more branches of a fluid conduit system or network (e.g., the branches connecting each of the bottles or other containers 110A, 110B, 110C, 110D of a manifold system 100 to the main conduit 116 in FIGS. 2A, 2B, 3A and 3C). In some embodiments, such branches or conduit segments can include one or more flow meter devices 114A, 114B, 114C, 114D, as illustrated schematically in FIG. 3C. Accordingly, in some configurations, the flow through a branch or conduit can be modified (e.g., continuously, intermittently, etc.) to achieve a desired or required treatment material mix being delivered from a manifold or other fluid source to a handpiece.

In some arrangements, a 3-way valve or other flow switching device 126 is positioned along the conduit 116 that fluidly couples the manifold assembly 100 to a handpiece assembly 50. As illustrated in FIG. 2A, such a 3-way valve 126 can be positioned downstream of the flow meter 120. However, in other arrangements, a 3-way valve can be positioned upstream of a flow meter, either in addition to or in lieu of placing such a valve downstream of a flow meter, as desired or required. The 3-way valve 126 can be coupled to an outlet conduit or other outlet fluid line 130 that extends to an exterior of the tower assembly 12 or other housing (e.g., to a conveniently positioned location (e.g., with direct and/or unobstructed access) of the tower assembly or other outer surface). For instance, the outlet conduit 130 can extend to a port or other coupling 132. Such a port or coupling can include a standard type of port or coupling 132. However, in alternative embodiments, the port or coupling 132 is non-standard (e.g., includes a custom design, shape, connection method, etc.).

According to some embodiments, the port or other coupling 132 is configured to receive a second, different handpiece assembly (e.g., a handpiece that is not the primary handpiece assembly 50). In other words, the port 132 can be coupled to a handpiece that is separate and distinct from the primary handpiece 50 (e.g., which is coupled to the manifold assembly 100 of the system). In some embodiments, such a different handpiece assembly (not shown) is configured to be placed in fluid communication with the bottles or other fluid containers secured to the manifold assembly 100. This can be accomplished by manipulation of the valve 126. For example, the valve 126 can be positioned to selectively permit treatment fluid to be delivered to either the primary handpiece assembly 50 or to the port 132. A handpiece assembly can be coupled to the port 132 to permit such a handpiece assembly to be placed in fluid communication with the manifold assembly. Thus, in some embodiments, as illustrated schematically in FIG. 2A, a system can include the ability to hydraulically couple a manifold assembly (and/or any other fluid source) to at least two handpieces.

The three-way valve 126 can be configured to be manually operated (e.g., by a user manipulating a switch, valve or other actuator). Alternatively, the valve 126 can be automatically controlled by the system (e.g., a control module of the system, gesture controls, etc.). Such automatic control can be based, at least in part, on one or more of the following: a treatment protocol being performed, one or more preferences or other inputs of the user (e.g., via a touchscreen or other user input device) and/or the like.

Accordingly, in some embodiments, the system can facilitate the use of two or more handpiece assemblies that can be hydraulically coupled to a manifold assembly. In other embodiments, the valve 126 can be configured to include more than two flow options. Thus, a valve can permit the flow of treatment fluid to three, four or more locations (e.g., handpiece assemblies, ports for receiving handpiece assemblies, etc.), as desired or required.

As discussed herein with reference to FIG. 3A, in some embodiments, treatment liquids and/or other fluids or materials contained in bottles or other containers 110A, 110B, 110C, 110D are transferred to a handpiece assembly 50 when a vacuum or suction source V is activated. For example, treatment fluid and/or other material included in one or more of the bottles or other containers 110A, 110B, 110C, 110D secured to the manifold assembly 100 can be transferred to the main fluid conduit 116 when the tip or other distal end 52 of the handpiece assembly 50, 60 is positioned on a skin surface to be treated when suction is generated along the tip or distal end 52 by activation of the vacuum or suction source V. As discussed, the tip or other distal end portion of a handpiece can include a peripheral lip or member to provide at least a partial seal with adjacent skin tissue during use, thereby creating an interior that is subjected to the vacuum or suction force generated by a vacuum or suction source. Thus, in such arrangements, treatment fluids and/or other materials from the bottles or other containers 110A, 110B, 110C, 110D are “pulled” or moved to the handpiece assembly 50 using suction created along the handpiece assembly.

In other embodiments, treatment fluids and/or other materials can be transferred to the handpiece assembly 50 from the manifold assembly 100 using the application of positive pressure within or along the main fluid conduit 116 and/or the bottles or other containers 110A, 110B, 110C, 110D secured to the manifold assembly 100. This can be accomplished either in lieu of or in addition to the generation of suction along the handpiece assembly 50 (e.g., as schematically depicted in FIGS. 3A and 3B and discussed above).

Fluids and/or other treatment materials from two or more bottles or other containers 110A, 110B, 110C, 110D secured to a manifold assembly 100 can be delivered to the handpiece assembly 50 sequentially or simultaneously, as desired or required. As discussed, the fluid network of the system can include one or more valves, other flow control devices or member, flow meters and/or the like to help regulate the flow of fluids and/or other materials from a fluid source (e.g., a manifold assembly, a vial or cartridge, etc.) to the tip or distal end of a handpiece.

According to some embodiments, the system is configured to deliver treatment fluid from the manifold assembly 100 to a handpiece assembly 50 continuously. However, in other embodiments, treatment fluid can be provided using a pulsed pattern. In some arrangements, a pulsed pattern can be created by selectively actuating one or more air-release valves 124 in fluid communication with the fluid conduit or line that couples (e.g., fluidly couples or otherwise connects) the handpiece assembly (e.g., handpiece) to the manifold assembly. The terms “handpiece assembly” and “handpiece” are used interchangeably herein.

With continued reference to FIG. 2A, the system 10 can comprise (or can be configured to be used with) one or more additional handpiece assemblies (e.g., handpieces) 60, 70 that couple to the tower or console 12. As illustrated, an alternative handpiece assembly 60 can include a handpiece that is not hydraulically coupled to the manifold assembly 100, and thus, not in fluid communication with any bottles or other containers 110 secured thereto. Instead, the alternative handpiece assembly 60 can include a recess or other area configured to receive a vial or other fluid container. The vial or other fluid container can include treatment fluids and/or materials that can be selectively delivered to the tip or distal end of the alternative handpiece assembly 60 (e.g., upon the activation of the vacuum source V).

According to some embodiments, the system is configured to advantageously accommodate both a handpiece 50 that is in fluid communication with a manifold assembly 100 and a handpiece 60 that is fluidly separated from the manifold assembly 100. Regardless of their exact configuration, any handpiece assemblies (e.g., handpieces) 50, 60 included in and/or used with a system 10 can be configured to be placed in fluid communication with the vacuum source V (e.g., vacuum pump positioned within the tower or console 12).

As illustrated schematically in FIG. 2A, suction conduits 140, 142 of the primary handpiece assembly 50 and the alternative handpiece assembly 60 can be fluidly connected to a 3-way valve or other flow control device 146. In some arrangements, such a valve 146 and at least a portion of the conduits or other fluid lines 140, 142, 148 connected to the valve 146 are positioned within a housing or other interior location of a tower or console 12.

According to some embodiments, an output conduit or fluid line 148 coupled to the three-way valve 146 is transferred (e.g., directly, indirectly) to one or more waste canisters 150. In FIG. 2A, the waste canister 150 is positioned along, at least partially, an exterior of the tower or console 12. In alternative arrangements, however, the waste canister 150 can be positioned at least partially within the tower or console 12 and/or any other location, as desired or required. In some embodiments, the waste canister is separate from the tower or console.

The waste canister 150 can be in fluid communication with the vacuum pump or other vacuum pump V of the system via one or more fluid conduits 160, valves 170, regulators and/or components. In some arrangements, the waste canister 150 comprises a lid adaptor 152 can includes a port, coupling or other connection to which the conduit 160 can be coupled.

With continued reference to FIG. 2A, the 3-way valve 146 to which both the primary handpiece assembly (e.g., handpiece) 50 and an alternative handpiece assembly (e.g., handpiece) 60 are hydraulically coupled can be automatically and/or manually controlled or operated. According to some embodiments, the valve 146 is automatically actuated or otherwise moved to a desired orientation based on one or more selections made by a user of the skin treatment system 10. For example, if a user selects a treatment procedure that uses treatment fluids from one or more of the bottles or other containers 110 secured to the manifold assembly 100, the control module of the system 10 can be configured to switch the three-way valve 146 to a position so that vacuum is generated in the fluid conduit 140 coupled to the main handpiece assembly (e.g., handpiece) 50. Alternatively, if a treatment protocol requires the use of a vial or cartridge that is secured to the handpiece itself, the three-way valve 146 can be automatically actuated to create suction within the fluid conduit 142 coupled to the alternative handpiece assembly (e.g., handpiece) 60.

The system 10A, 10B can be configured to accommodate one or more other types of handpiece assemblies and/or other modalities (e.g., besides a primary and a secondary/alternative handpiece, as discussed herein). For example, as illustrated schematically in FIG. 2A, a lymphatic handpiece assembly (e.g., handpiece) 70 can be secured to a port or other coupling (not shown) of the tower or console 12. Once coupled to the tower or console 12, such a handpiece assembly 70 can be placed in fluid communication with the system's vacuum pump or other vacuum source V (e.g., via one or more conduits, filters, valves, mufflers and/or any other hydraulic components).

In some embodiments, as illustrated schematically in FIG. 2B, the tower or console 12 of the system 10B, comprises only a single port, coupling or other connection point configured to secure to a handpiece 50. As shown, unlike the embodiment of FIG. 2A, the need for a separate (e.g., second) port or coupling 132 can be eliminated, if desired or required. Such a configuration can simplify the overall system, reduce the complexity of the hydraulic system within the tower or console, improve manufacturability and provide one or more other advantages or benefits.

With reference to FIG. 2B, the tower or console 12 can include a manifold assembly 100. As discussed above with reference to the embodiment depicted in FIG. 2A, the manifold assembly 100 can be configured to receive one, two or more (e.g., three, four, five, more than five, etc.) containers (e.g., bottles) 110A-110D. Fluids and/or other treatment materials from one or more containers or other sources coupled to a manifold assembly can be selective delivered to the tip or distal end of a handpiece that is coupled (e.g., directly, indirectly, fluidly, etc.) to the manifold assembly and/or other portions of the tower or console of the system.

In some arrangements, as illustrated schematically in FIG. 3A, one or more valves or other flow control devices or components 112 can be positioned between each fluid source (e.g., a bottle) and a main delivery conduit or other fluid line 116 that places the manifold assembly 100 in fluid communication with a handpiece assembly 50. The valves or other flow control devices 112, which can include 2-way valves, check valves and/or any other type of valve, can be configured to regulate the flow of treatment materials from one or more of the bottles or other containers 110A, 110B, 110C, 110D to the main delivery conduit 116, and thus, to the handpiece assembly 50. For any of the embodiments disclosed herein or variations thereof, including, for instance, the arrangement schematically illustrated in FIG. 3A, one or more flow meters or other flow measuring or estimating devices can be included to help determine if and how to alter flow through one or more portions of the hydraulic network of the system (e.g., the manifold assembly, the conduit that hydraulically couples the manifold assembly to a handpiece, fluid passages of the handpiece, etc.).

As noted above with reference to FIG. 3A, the handpiece assembly 50 can be placed in fluid communication with a suction source (e.g., vacuum pump, not shown) using one or more vacuum or suction conduits or other fluid lines 140. In the arrangements disclosed herein, for example, the manifold assembly 100 comprises a total of four fluid branches. However, a system can comprise more or fewer fluid branches (e.g., 1, 2, 3, 4, 5, 6, 7, 8, more than 8, etc.), as desired or required by a particular application or use. Further, one or more of the bottles or other containers 110A, 110B, 110C, 110D that are positioned within the manifold assembly 100 can include a cleaning solution (e.g., for the periodic flushing and cleaning of the manifold assembly).

According to certain embodiments, one or more of the fluid conduits of the manifold system illustrated in FIG. 3A are configured to provide a serum, other treatment fluid and/or the like. Alternatively, however, one or more of the conduits can be configured to receive water (e.g., distilled, tap water, filtered, etc.), saline, other dilutants or dissolvents, other fluids and/or the like to the handpiece assembly 50. Such fluids can be adapted to contact and dissolve, dilute, liquefy, soften and/or otherwise mix with one or more solids, gels and/or other materials positioned within or on various surfaces or portions of the handpiece assembly 50 (e.g., tip). This can provide a convenient method of providing one or more materials at the skin-tip interface and/or any other location where such materials are desired or required.

Further, as illustrated schematically in FIG. 3B and discussed in this application, for any skin treatment system 10A, 10B disclosed herein or equivalents thereof (e.g., the embodiment of depicted in FIG. 2A and/or FIG. 2B), the system can be configured to permit a user (e.g., an aesthetician, a cosmetician, a dermatologist, etc.) to (1) connect a handpiece assembly 50 to a manifold assembly 100 (e.g., using a “dummy” cartridge 90, as shown in FIG. 3A), and/to (2) insert an actual fluid or treatment material-containing cartridge 92 directly into the handpiece assembly, e.g., a recess of the handpiece assembly (as shown in FIG. 3B). These two options can be used interchangeably to perform a treatment procedure or protocol, as desired or required.

With continued reference to the schematic of FIG. 2B, a fluid conduit 116 can place the manifold assembly 100 (and thus, any bottles or other containers 110A-110D secured thereto) in fluid communication with the handpiece assembly 50. In the illustrated embodiments, the system 10B includes a flow sensor 120, a check valve 122, a purge valve 124 and a backflow prevention valve 126 along the fluid conduit or fluid pathway from the manifold assembly 100 to the handpiece assembly 50. However, in other arrangements, more or fewer (and/or different fluidic components, mechanical components, etc.) can be included. Such components can be positioned and/or otherwise included, at least partially, within or along the fluid pathway or fluid conduit (e.g., within and/or outside the tower or console 12) and/or within or near the handpiece assembly (e.g., handpiece) 50, as desired or required.

As discussed above with reference to the schematic illustrated in FIG. 2A, in some arrangements, the vacuum or suction system or source that is configured to be coupled to the handpiece 50 in FIG. 2B can be identical, similar or substantially similar, to the vacuum or suction system or source for a separate handpiece (e.g., handpiece 60) using a different port or connection point. In some embodiments, the tower or console (and/or the fluid components of the tower or console and/or the fluid components of the entire fluid network of the system) are configured to use (and actually do use) the identical vacuum or suction system or source. The one or more suction or vacuum systems or sources that help create a vacuum or suction pressure to any handpieces fluidly coupled to the system can be at least partially coupled to or incorporated within the tower, console or other portion or component of the system. However, in other embodiments, such a vacuum or suction system or source is separate from the system, but configured to operatively couple to the system to help generate the desired or required negative pressure (e.g., relative to atmospheric or ambient pressure) along the handpiece-skin interface during use.

An output conduit or fluid line 148 coupled to the three-way valve 146 can be directed to one or more vacuum waste canisters 150. In FIG. 2A, the vacuum waste canister 150 is positioned along an exterior of the tower or console 12. In alternative arrangements, however, the waste canister 150 can be positioned at least partially within the tower or console 12 and/or any other location, as desired or required. For example, in some embodiments, a waste canister 150 can be in fluid communication with the vacuum pump or other vacuum pump V of the system via one or more fluid conduits 160, valves 170, regulators and/or components. In some arrangements, the waste canister 150 comprises a lid adaptor 152 that includes a port, coupling or other connection to which the conduit 160 can be coupled.

In some embodiments, as schematically illustrated in FIG. 2B, the system 10B is adapted to accommodate one or more other types of handpiece assemblies and/or other modalities. For example, as illustrated schematically in FIG. 2B, a lymphatic handpiece assembly 70 can be secured to a port or other coupling (not shown) of the tower or console 12. Once coupled to the tower or console 12, such a handpiece assembly (e.g., handpiece) 70 can be placed in fluid communication with the system's vacuum pump or other vacuum source V (e.g., via one or more conduits, filters, valves, mufflers and/or any other hydraulic components).

In embodiments where the tower or console 12 of the system 10B includes only a single port, coupling or other connection for the handpiece assembly 50 (e.g., as in the arrangement of FIG. 2A), the handpiece 50 can be configured to receive one of a plurality of tips 52 along its distal end. This can help with accommodating various treatment modalities that would only otherwise be available with the use of two or more different handpiece assemblies 50 (e.g., such as the arrangement schematically illustrated in FIG. 2A).

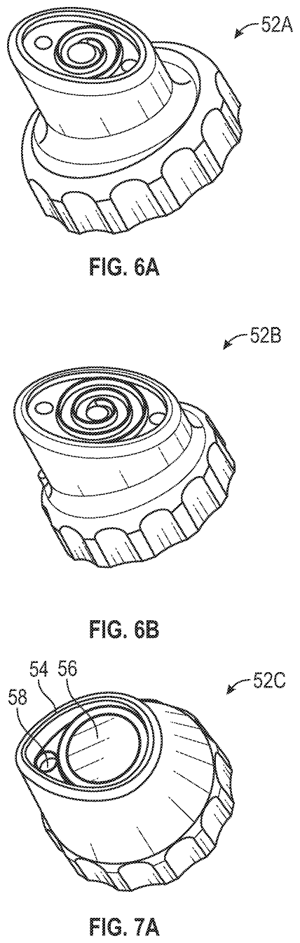

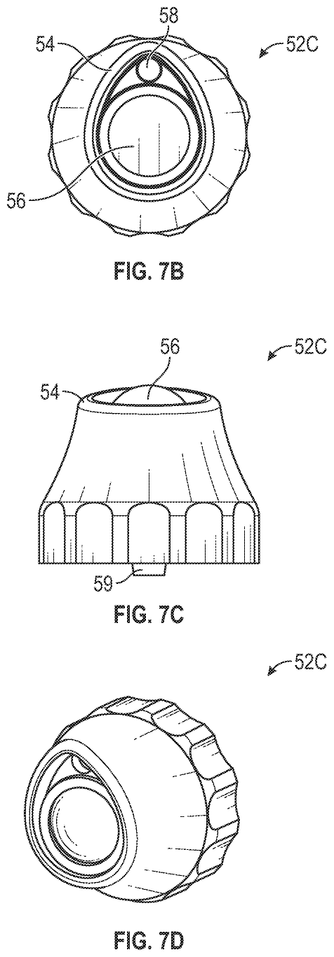

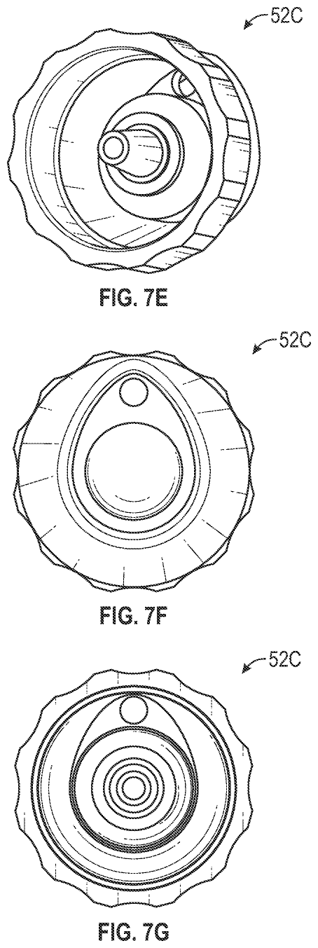

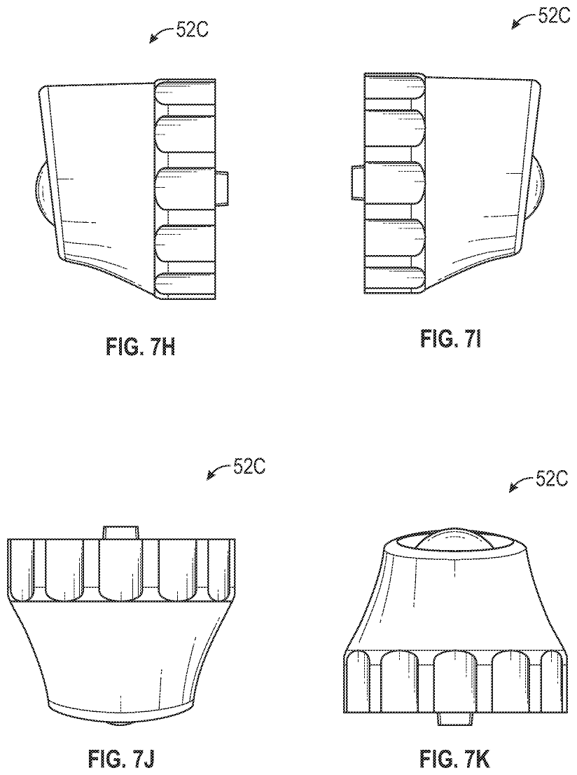

Various embodiments of tips 52, 52A, 52B, 52C that may be secured to the distal end of a handpiece 50, 60, 70, such as for example, any of the handpieces disclosed or contemplated herein or equivalents thereof. Non-limiting example embodiments of such tips are illustrated in FIGS. 6A, 6B and 7A to 7C. Additional information regarding tips is provided below.

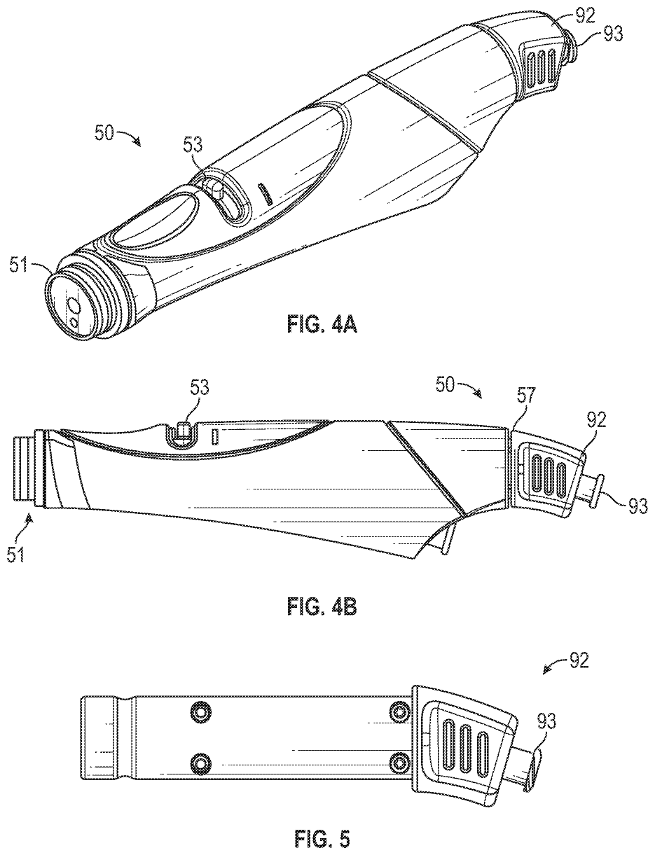

One embodiment of a handpiece or handpiece assembly 50 that can be used with the tower or console 12 of the skin treatment system 10A, 10B is illustrated in FIGS. 4A and 4B. As shown, the handpiece 50 can include a lever or other controller 53 for adjusting the flow of treatment fluids and/or materials to the distal end 51 of the handpiece. A tip 52 can be removably secured to the distal end 51 of the handpiece.

With continued reference to FIGS. 4A and 4B, the handpiece or handpiece assembly 50 can be configured to receive a cartridge 92. In some embodiments, as discussed below with reference to FIG. 3B, the cartridge or vial 92 itself is configured to store a treatment liquid or other material (e.g., within one or more of chambers of the cartridge 92). However, in other arrangements, as illustrated in FIG. 3A, the cartridge or vial 92 can be a “dummy” cartridge or an interface device or component that places the handpiece in fluid communication with a manifold assembly or other fluidic network 100 (e.g., of a tower or console 12 of the skin treatment system 10A, 10B). In such embodiments, the cartridge 92 includes a proximal coupling (e.g., luer) 93 that can receive a fluid conduit (e.g., the main delivery conduit or other fluid line 116) that places the manifold assembly 100 in fluid communication with the handpiece assembly 50.

According to some embodiments, the cartridge 92, regardless of whether it is a cartridge that comprises fluid or other treatment material within an interior chamber or whether it is a “dummy” cartridge 92 that places the handpiece in fluid communication with a manifold or other fluid source or network, is configured to be removably positioned within a recess 57 of the handpiece assembly 50. In some embodiments, the recess comprises an open recess that is accessible along the exterior of the handpiece assembly. In some arrangements, the recess 57 is easily accessible from the exterior of the handpiece assembly to permit a user to insert and remove a cartridge 92 within/out of the recess without manipulating any portion of the handpiece assembly 50 (e.g., without opening an interior of the handpiece, without taking any additional steps, etc.), as desired or required. In some configurations, the recess 57 is located along the proximal end of the handpiece assembly 50 and faces in an opposite or substantially opposite direction than the distal end 51 and any tip 52 secured along the distal end 51 of the handpiece assembly 50.

For any of the skin treatment system embodiments disclosed herein, a cartridge 92 can be configured to be locked and unlocked to the handpiece assembly 50. In some arrangements, the cartridge 92 can be locked and unlocked using a rotation, twisting, pushing, sliding and/or other movement of the cartridge relative to the handpiece once the cartridge is secured to the handpiece. According to some embodiments, the cartridge 92 is configured to lock to the main body portion of the handpiece assembly using one or more devices or methods, such as, for example, locking tabs, clasps, magnetic connectors, other fasteners and/or the like.

With continued reference to FIGS. 4A and 4B, a luer or other coupling 55 located along the proximal end (e.g., proximal half) of the handpiece assembly 50 can be configured to couple to a vacuum or suction conduit 140 (see, e.g., FIGS. 2A and 2B). As illustrated in FIG. 3A and discussed herein, the suction conduit or line 140 once fluidly coupled to a vacuum or suction source (e.g., vacuum pump) V can help create a vacuum or suction force along the distal end and/or tip 52 of the handpiece. As also discussed herein, the tip or other distal end of a handpiece used in the system can include a peripheral lip or member that contact skin tissue during use. In some embodiments, such a peripheral lip or member extends along the outer radial boundary of a tip or distal end and defines an interior. Such an interior can be in fluid communication with one or more vacuum and/or fluid delivery conduits (e.g., via corresponding ports or openings). Accordingly, once the tip or other distal portion is positioned along a targeted skin tissue, a closed seal can be formed or substantially formed between the tip/distal end and the skin surface. Consequently, the vacuum or suction force can facilitate the transfer of fluid and/or other treatment materials to tip (e.g., from the cartridge secured to the handpiece, from containers of manifold system placed in fluid communication with the handpiece, etc.).

FIG. 3A schematically illustrates the use of a “dummy” cartridge 90 secured to the handpiece 50. Such a cartridge 90 can be placed in fluid communication with a manifold system 100, e.g., a manifold system 100 of a tower or console system. In some arrangements, the cartridge 90 includes a port or other connector (e.g., a luer) 93 along its proximal end that is sized, shaped and otherwise configured to connect to a conduit 116 that is in fluid communication with the manifold system 100.

In other embodiments, the “dummy” cartridge 90 of FIG. 3A can be replaced with a fluid and/or other treatment material cartridge 92, as illustrated in FIG. 3B. Such a cartridge 92 can include one or more chambers that contain a serum and/or other treatment fluid or material. In such embodiments, the vacuum or suction force generated along the tip or distal end of the handpiece can help draw serums and/or other treatment fluids/materials stored within the cartridge to the tip or distal end when the tip or distal is positioned along the targeted skin surface.

In some embodiments, both a “dummy” cartridge 90 that is in fluid communication with a manifold 100 (e.g., as schematically illustrated in FIG. 3A) and a cartridge 92 that itself includes one or more serums or fluids, e.g., a booster vial (e.g., as schematically illustrated in FIG. 3B) can be used in a particular system and/or procedure. Therefore, in some arrangements, the user can swap cartridges 90, 92 during and/or between treatment steps or procedures, as desired or required.

As illustrated in FIGS. 2A and 2B, one or more flow meters one or more flow meters 120 or other flowrate measuring devices can be included in the fluid path placing the manifold assembly 100 in fluid communication with one or more handpiece assemblies 50, 60. The flow meter 120 can be used to measure the amount of fluid (e.g., treatment fluid, cleaning or rinsing solution, etc.) that is being transferred from bottles or other containers coupled to the manifold assembly 100. As discussed, this can help confirm the proper and/or safe function, operation and/or use of the skin treatment system 10. For example, the flow meter 120 can help ensure that an accurate amount of treatment fluid is being delivered to a handpiece assembly. The use of flow meters 120 can help identify leaks, misuse and/or the like. In some embodiments, the flow meter 120 comprises an electromechanical flow meter. However, any type of flow meter 120 can be used, including, without limitation, a positive displacement flow meter, a velocity flow meter (e.g., turbine, paddle wheel, ultrasonic, electromagnetic, vortex-shedding, ultrasonic flow meters, etc.), mass flow meters or any other type of flow meter, as desired or required.

With reference to the schematic embodiments illustrated in FIGS. 2A and 2B, the system 10A, 10B can include at least one flow meter 120 that is designed and otherwise configured to measure the flowrate of treatment fluid being transferred from the manifold assembly 100 (and one or more bottles or containers 110A-110D secure thereto) to the handpiece assembly 50. As shown, the flow meter 120 can be positioned between the entire manifold assembly 100 (e.g., fluidly downstream of the a collective output of the manifold assembly, downstream of the point where flow from all bottles or other containers 110A-11D of the manifold assembly is combined, etc.). Therefore, in some embodiments, the system 10A, 10B is configured to detect and measure (e.g., within a desired level of accuracy, more example within plus or minus at least 1%, 5%, 10%, 15%, 20%, percentages greater than 20%, percentage values between the foregoing ranges and values, etc.) a flowrate of all fluids and/or other treatment materials exiting the manifold assembly 100 at any point in time during a procedure.

As noted herein, the ability to detect fluid and/or other treatment material flowrates can provide one or more benefits and/or advantages. For instance, measuring (e.g., measuring flowrate within a desired accuracy range, approximating flowrate on a more general and/or broad level, etc.) can help track, predict/approximate the use of materials, reusable, supplies and/or the like (e.g., for reordering, misuse, etc.). Further, the measurement or approximately of flowrate can help identify leaks, misuse and/or the like.

In some embodiments, the use of flow metering can help with properly balancing the volume of fluids being delivered from a bottle or other container secured to a manifold system or other fluid network to a handpiece. This can be especially helpful when two or more fluids and/or other treatment materials are being delivered simultaneously from the manifold assembly to the handpiece. As noted herein, the use of valves or other flow control devices, flow metering devices, sensors and/or other components can be used to accomplish a desired amount of flow control and balancing when the system is in use.

By way of example, in some embodiments, a system can include one or more other flow meters 114A-114D, either in lieu of or in additional to a flow meter 120 that is configured to measure or approximate a total flowrate (e.g., combined from all bottles, containers or other sources 110A-110D) of fluids and/or other treatment materials being transferred from the manifold assembly 100 to the handpiece 50. Such flow meters 114A-114D, as depicted schematically in FIG. 3C, can be configured to measure the flowrate of fluids and/or other treatment materials being transferred out of each bottle or other container 110A-110D secured to the manifold assembly.

Accordingly, in some embodiments, this configuration can allow for the monitoring of the amount (e.g., volume, mass, etc.) of treatment fluid and/or other materials originating from each bottle or other container 110A-110D secured to the manifold assembly 100 (e.g., toward the outlet of the manifold assembly 100).

In some embodiments, one or more valves or other flow-control features or devices (not illustrated herein) can be positioned adjacent (e.g., fluidly, mechanically, actually and/or the like) each of the flow meters 114A-114D. Such valves or other flow-control devices or features can be configured to be automatically and/or manually adjusted. In some embodiments, automatic adjustment of the valve position (and thus, the flowrate of the fluids and/or other treatment materials passing past the valve or other flow-control device) can allow for the proper balancing (e.g., automatically) of the one or more fluid/material streams being transferred from one or more containers 110A-110D secured to the manifold assembly (e.g., fluidly to or in the direction of the handpiece assembly).

According to some arrangements, for example, an automatic flow control or balancing configuration can include one or more of the following: a control unit (e.g., a processor memory, etc.), a controller, a sensor, wiring, wireless communication components, a user input device, a user output device and/or the like, in addition to any fluid conduits or lines, couplings, flow meters 120, 114A-114D and/or valves or other flow-control features and/or devices.

Therefore, in some embodiments, two or more fluids and/or other treatment materials can be delivered form containers 110A-110D secured to a manifold assembly to the handpiece assembly 50. The ability to combine fluids such that they are simultaneously or substantially simultaneously delivered to the handpiece can provide one more advantages or benefits. For example, such arrangements can allow for the activation (e.g., short-term, long-term, etc.) one more fluid/material being delivered by another fluid/material being delivered. In other embodiments, the combination of materials (e.g., two, three or more) can create a new or variant of a treatment fluid or material and/or provide some other benefit. Thus, customized formations can be created by regulating or otherwise controlling the relative proportions of two or more fluids and/or other treatment materials being delivered to the handpiece assembly (e.g., handpiece) and the tip or other distal end or portion of a handpiece. Such relative combining can be performed automatically or manually, as desired or required.

In some embodiments, such embodiments can help determine, and if/when necessary, adjust the flowrates and/or other delivery rates of fluids and/or other materials from the one or more containers (e.g., bottles) secured to the a manifold assembly to the handpiece assembly 50. This can ensure that the proper amount of material is being provided to a subject when executing and/or having executed a skin treatment procedure. Such embodiments can improve efficacy and/or treatment results (e.g., for the subject being treated), can help improve efficiency and/or reduce costs, can help with the recordkeeping and/or inventory management for a user, customer, seller, etc. and/or the like.

As noted above, a handpiece assembly 50 configured to be used with one or a variety skin treatment tips that can be removably and/or replaceably positioned along a distal end of handpiece assembly (e.g., handpiece). At least some embodiments of such tips 52, 52A-52C are illustrated in FIGS. 6A, 6B and 7A to 7C. As shown, the tips can include any tips disclosed in U.S. Pat. No. 8,048,089, filed as U.S. patent application Ser. No. 11/392,348 on Mar. 29, 2006 and issued on Nov. 1, 2011, the entirety of which is hereby incorporated herein and made a part of this specification. See, for example, FIGS. 5A to 11E and the corresponding disclosure, in U.S. Pat. No. 8,048,089.

FIGS. 7A to 7C illustrate different views of one embodiment of a tip 52C configured to be removably positioned along a distal end of a handpiece assembly 50. As shown, the tip 52C can include a peripheral or outer lip 54 along its distal portion. Such a peripheral or outer lip 54 can define an interior (e.g., interior region). According to some embodiments, the interior region can include a rollerball 56 and/or similar feature. In some embodiments, the rollerball can be in fluid communication (e.g., direct, indirect) with an interior region of the tip. The interior region of the tip can be in fluid communication with and/or otherwise configured to come in contact with a treatment serum, fluid and/or other material that is configured to be delivered to the tip (e.g., along an exterior of the rollerball).

With continued reference to FIGS. 7A to 7C, the tip 52C can further include one more ports or other openings 58. Such ports or other openings 58 can help place the tip in fluid communication with a vacuum or other suction source and/or a fluid source (e.g., a canister, bottle and/or other container, regardless of whether or not such a source is positioned with a manifold assembly).

In some embodiments, the top (e.g., most distal portion) of the rollerball 56 is aligned with or substantially aligned with the top (e.g., most distal portion) of the peripheral lip 54. In other embodiments, the top (e.g., most distal portion) of the rollerball 56 is proximal to or substantially proximal to the top (e.g., most distal portion) of the peripheral lip 54. In alternative configurations, the top (e.g., most distal portion) of the rollerball 56 is distal to (e.g., “proud”) or substantially distal to the top (e.g., most distal portion) of the peripheral lip 54.

In some embodiments, the top (e.g., most distal portion) of the rollerball 56 can be proximal or distal to the top (e.g., most distal portion) of the peripheral lip 54 by 0% to 25% (e.g., 0-25, 0-5, 5-10, 10-15, 15-20, 20-25, 5-20%, greater than 25%, etc.) of the diameter of the rollerball 56. In some embodiments, the top (e.g., most distal portion) of the rollerball 56 can be proximal or distal to the top (e.g., most distal portion) of the peripheral lip 54 by 0.1 to 10 mm (e.g., 0.1-10, 0.1-1, 1-2, 2-3, 3-4, 4-5, 0.1-5, 5-10 mm, values between the foregoing ranges or values, greater than 10 mm, etc.) relative to the top of the rollerball 56.

According to some embodiments, the use of a rollerball 56 along a tip can facilitate the delivery of fluids and/or other treatment materials as the tip (and thus the rollerball) is moved along a targeted skin surface. In some embodiments, the peripheral lip 54 can help engage the tip to targeted skin tissue and form or create a “seal” between the tip and skin tissue. In some arrangements, once such a “seal” has been formed, any vacuum or suction created along the ports or other openings 54 can help remove spent fluids/materials, exfoliated skin tissue and/or other debris and/or the like away from the tip 52C. The creation of suction or vacuum along the tip can also assist with the delivery of fluids to the tip (e.g., from a manifold, a separate cartridge secured to a handpiece, etc.). The delivery of fluids could occur through/around the rollerball 5, through one or more ports or openings and/or the like, as desired or required.

FIGS. 7D to 7K illustrate different views of one embodiment of a tip 52C having a rollerball identical or similar to the embodiment depicted in FIGS. 7A to 7C. FIGS. 7D and 7E show front and rear perspective views, respectively, of the tip. FIGS. 7F and 7G show front and rear views, respectfully, of the tip. FIGS. 7H and 71 are left side and right side views, respectfully, of the tip. FIGS. 7J and 7K are top and bottom views, respectfully, of the tip.

Further, in some arrangements, the tip can include one or more structures, features, members and/or other components that are configured to at least partially abrade tissue when the tip is moved relative to skin. In some embodiments, such features have sharp or substantially sharp features to assist with abrading skin tissue. In some arrangements, such features are stationary and are incorporated in the tip (e.g., as part of a unitary structure with the tip). In some embodiments, the abrading structure(s) is/are positioned along an interior defined by a peripheral lip (e.g., as illustrated in FIGS. 6A and 6B). In other embodiments, the peripheral lip comprises the abrading structure, at least in part, either in addition to or in lieu of any additional (e.g., interior) abrading structures or features.

As noted above, such tips 52, 52A, 52B, 52C can be shaped, sized and/or other configured to be secured to and/or removed from a handpiece assembly 50, 60, 70. The tips can be disposable so that they are discarded and/or replaced during or after a treatment procedure. However, in other arrangements, the tips can be configured (e.g., as result of its material(s) (e.g., stainless steel, other metals or alloys, other composites, etc.), as result of its construction or design, etc.) to be reusable. Thus, in some arrangements, the tips are configured to withstand the temperature variations, chemicals and/or other conditions to which they may be subjected for cleaning, disinfecting, sterilization and/or the like.

In some embodiments, a plane defined by the distal-most portion of the tip (e.g., the peripheral lip) can be angled relative to both the longitudinal axis of the tip (and the handpiece assembly to which is secures) and an orthogonal or perpendicular axis of the longitudinal axis. This can provide one or more advantages or benefits to a user who is manipulating a handpiece assembly, including increasing the surface area of such a plane, providing an improved ergonomics, functionality and comfort features and/or the like.

As noted above, the system can be configured to obtain information regarding the contents of a bottle or other container that is positioned within a corresponding receiving station of the manifold assembly.

FIG. 8A illustrates a manifold assembly 100 of a tower 12 or other portion of a skin treatment system 10. The manifold assembly 100 is configured to receive a plurality of bottles 210 (e.g., containing serums, treatment fluids, cleaning solutions, etc.). In some embodiments, as depicted in FIG. 8B, each of the bottles 210 can include a main body portion 214 and a cap portion 216. The cap portion 216 can be configured to include at least one surface or area that is sized, shaped and otherwise arranged to receive an automatic identification tag 300, such as, for example, a RFID tag or chip, a barcode, etc. Such tags can be used to advantageously store information regarding the specific bottle, vial or other container. For example, the tag can include information regarding the contents of the container, expiration date, manufacturing date, size, lot number, skin procedure with which the contents are intended to be used, other limitations or restrictions on use (e.g., counter-indications, adverse effects, other fluids with which the contents should not be combined, etc.).

The RFID or other identification tag can be read or otherwise detected (e.g., automatically, manually, etc.) by one or more readers or detectors 104 of a manifold assembly 100, a handpiece assembly and/or any other portion of the tower or console and/or the skin treatment system. For example, in some embodiments, such a reader 104 can be placed at or near each station of a manifold system 100 (e.g., adjacent the portion of the manifold to which the nozzle or top portion of the bottle or other container 210 secures). Accordingly, the RFID or other type of reader 104 can detect and identify the RFID tag of the bottle or other container.

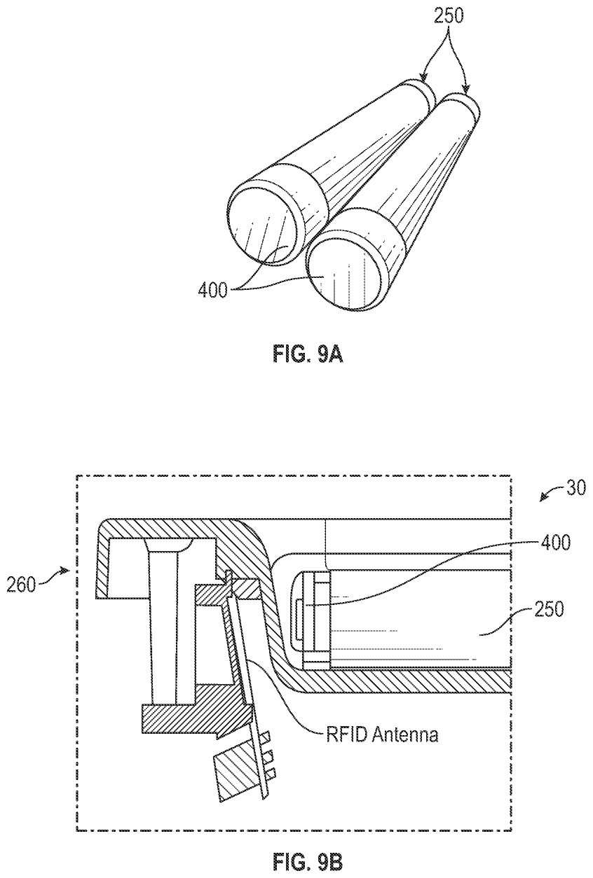

Likewise, as illustrated in FIGS. 9A and 9B, a RFID or other type of reader 260 can detect and identify the RFID or other identification tag 400 of a vial 250 when such a vial or smaller container 250 is positioned within or near the reader 260. In some embodiments, as depicted in FIGS. 1 and 9B, the tag reader 260 for vials and similar smaller fluid containers 250 is positioned in or near the tray 30 of the console or tower 12.

According to some embodiments, the tray or other storage or working platform 30 includes specially formed recesses that are shaped, sized and otherwise configured to receive vials or other smaller containers or containers 250. In some embodiments, such vials or containers are sized, shaped and/or otherwise adapted to hold up to 5 ml to 20 ml (e.g., 5 ml, 6 ml, 7 ml, 8 ml, 9 ml, 10 ml, 11 ml, 12 ml, 13 ml, 14 ml, 15 ml, 15 ml to 20 ml, 5 ml to 10 ml, 8 ml to 12 ml, capacities between the foregoing values and ranges, etc.) of fluid and/or other treatment materials that may be used as part of a treatment procedure and/or other procedure in connection with the system (e.g., cleaning, rinsing, priming, other preparatory actions or steps, etc.). However, in other embodiments, the capacity of such vials can be less than 5 ml or greater than 20 ml, as desired or required.

In some arrangements, the vials or other small containers 250 are configured to be positioned within such recesses or other receiving areas in a way that places the RFID tag or other identifier of each vial 250 in a desired orientation relative to the RFID or other type of reader 260. Therefore, placement of a vial within such a recess of the tray 30 (and/or removal of a vial from a recess) can automatically allow the reader 260 to obtain information regarding the vial.

In circumstances where the detected identifier is inconsistent with the proper, predicted, safe, appropriate and/or approved operation of the system, the system can be configured to prevent fluid from that vial or container from being used (e.g., by terminating the vacuum source, by maintaining a solenoid valve or other valve in the closed position, by otherwise preventing the flow of fluid from one or more of the bottles or other containers to the fluid delivery system of the manifold 100, the handpiece and other portions of the tower and/or the skin treatment system, etc.).

The use of the RFID or other identification tags on the bottles, vials and/or other containers of the system can provide one or more other advantages or benefits. The collection of data regarding use of and/or related to the corresponding container (e.g., bottle, vial, etc.) can be gathered or otherwise collected to generate reports for billing, reordering and/or other purposes. In some embodiments, the number of times that a container can be removed and reinserted within a manifold or handheld assembly can be limited (e.g., 1, 2, 3, 4, etc.), as desired or required. For example, such limits can help prevent or reduce the likelihood of contamination of the fluid. In some embodiments, the automatic identification of the fluid container being secured to the system (e.g., manifold station, handheld assembly, etc.) can allow the system to determine if a rinse, flush and/or other steps or actions are required before the fluid from that container can be used and/or any other action can be taken in connection with use of the system.

According to some embodiments, the use of RFID or other identification tags can facilitate the execution of a particular skin treatment protocol by the system. For instance, the system can include various bottles containing fluids necessary to carry out any one of a number of various skin treatment procedures. For example, a treatment sequence can be configured for use in procedures for periodic or normal microdermabrasion treatment, anti-aging, anti-acne, skin lightening, oily skin treatment and/or the like. Each of the sequences, protocols or modes can include the delivery of one, two or more various serums and/or other fluids that are housed in the bottles or other containers 210 in fluid communication with the manifold assembly 100 and/or other portion of the skin treatment system.

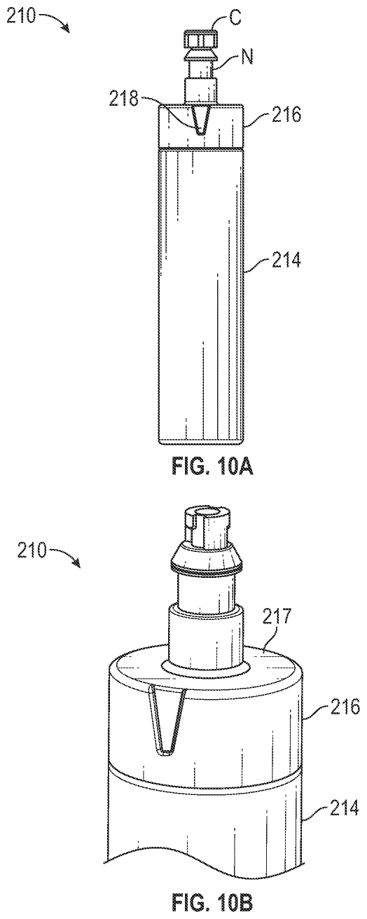

FIGS. 10A and 10B illustrate different views of one embodiment of a bottle 210 configured to be secured to a manifold assembly 100 of a skin treatment system 10. As shown, the bottle 210 can include a main body portion 214 and a cap portion 216. As noted above, the cap portion 216 can be configured to include at least one surface or area that is sized, shaped and otherwise arranged to receive an automatic identification tag 300, such as, for example, a RFID tag or chip, a barcode, etc. Therefore, in some arrangements, the cap 216 includes a generally planar annular section 217 for receiving a tag (not shown in FIG. 10B). As shown, the cap portion 216 can include a nozzle N that extends upwardly (e.g., away from the main body portion 214) and is configured to secure within a corresponding station or other portion of the manifold assembly 100. In some embodiments, the bottle cap portion 216 comprises one or more notches or other features 218 to assist with alignment of the bottle 210 relative to the manifold.

For any of the embodiments disclosed herein, a skin treatment system and/or the method being performed using a skin treatment system can incorporate one or more light treatment steps. According to some embodiments, the light source comprises a light wand, a panel and/or some other device or system. The light can include light emitting diodes (LEDs). The color (e.g., blue light, red light, etc.), intensity, duration and/or one or more other features related to the LEDs or other light source can be adjustable to accomplish a particular goal or purpose. For example, light therapy can assist in improving the result of treating skin for acne, aging, inflammation, etc. In some embodiments, the light source, which can include a device that is positioned and maintain a position over a subject's face or other portion of the anatomy, can be configured to operatively couple to a tower, console or other portion of a skin treatment system. The light source or other device can be directly or indirectly coupled to the tower or other portion or component of the system. In other embodiments, the light source or device is not directly or even physically coupled to the system.

In some embodiments, the light source can be configured to secure (e.g., removably), to be positioned on, along or near and/or otherwise to work with a tower or console of a skin treatment, such as, for example, the tower or console 10 illustrated in FIG. 1. The light source can include a want that is physically coupled (e.g., via a cable or other connecting line) to a tower or console (and/or any other components and/or portion of a skin treatment system). However, in other embodiments, the light source is not physically coupled to the tower or console (and/or any other component or portion of the system). In such configurations, the light source can operatively couple to the tower or console (and/or any other components or portion of a skin treatment system, e.g., a processor, a cloud network, etc.) wirelessly.

As noted, the light source can include a wand or other handpiece device. However, in other arrangements, the light source comprises a panel or other some device that is configured to be positioned over a subject's skin (e.g., like a tent or other covering member). Such a panel configuration can include one or more hinges or other components to allow the light source to fold and/or otherwise be reconfigured to a desired orientation or shape.

Regardless of the exact configuration of the light source, the light source can be configured to be charged/recharged by the tower or console (and/or any other component or portion of the skin treatment system). For instance, the tower or console can include a receiving area (e.g., charging base, docking station, an inductive charging area, etc.), a charging cable, a charging port and/or the like to which the light source can be operatively coupled. This can facility charging of the light source when not in use by the practitioner. In one embodiment where the light source includes a panel, the tower or console can include a receiving area along the rear (e.g., behind the display 20) for the light source.

In some embodiments, control of the light source can be integrated with other control features of the skin treatment system. For example, one or more properties of the LEDs or other type(s) of light of the light source can be adjusted (e.g., manually, automatically, etc.) using a main touchscreen 20 of the tower or console, using a separate device (e.g., a PC operatively coupled to the system, a smart device, etc.), as desired or required. The light source features described above can be incorporated into any of the skin treatment system/device/method embodiments disclosed herein.

According to some embodiments, a system (e.g., a touchscreen of a tower or console) can be controlled using gesture control technologies. Gesture control can be incorporated into a particular system either to replace or to supplement one or more other controls (e.g., a touchscreen or other user input device or component).

In some arrangements, the system includes one or more visual sensors, cameras and/or other gesture-control components along a frame of the touchscreen of the tower or console assembly. In one embodiments, four or more (e.g., 4, 5, 6, more than 6, etc.) visual sensors are included. These sensors and/or other components can be positioned and configured to detect hand motions of the individual using the system.

According to some embodiments, the system can include a total of four gesture sensors. For example, in such a configuration, two gesture sensors located on, along or near the top of the tower or console (and/or other portion) of the system, one gesture sensor on, along or near the left side of the tower or console (and/or other portion) of the system, and one gesture sensor on, along or near the right side of the tower or console (and/or other portion) of the system.

In some embodiments, the top sensors are used to move from one step to the next (or another) step within the user interface. In some arrangements, the left side and the right side gesture sensors are responsible can control another aspect of the treatment procedure, such as, for instance, increasing or decreasing the value of vacuum or suction being applied. Systems that incorporate gesture control can be customized so that the user or group of users can select what certain gestures modify with respect to the operation of the system. Regardless, the use of gesture control can permit a user to regulate or otherwise modify one or more aspects of the skin treatment system without the need to physically contact the system.

In some embodiments, the gesture sensors and/or other control aspects of the gesture control system can be activated only during certain steps of a treatment procedure. In some embodiments, two or more of the gesture sensors are configured to detect the same movements to control the same aspects of the system's operation. Thus, the system can advantageously include redundancy with respect to its gesture control system to ensure that it operates consistently and as intended during use.

Benefits of a gesture control system in connection with a skin treatment system include, but are not limited to, permitting hands-free operation of the system's user interface, allowing the user to keep gloves on during a treatment procedure while maintaining control, promoting cleanliness of the touchscreen and other system components, creating a more efficient treatment process and other benefits or advantages.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

While the inventions are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited. The methods summarized above and set forth in further detail below describe certain actions taken by a practitioner; however, it should be understood that they can also include the instruction of those actions by another party. The methods summarized above and set forth in further detail below describe certain actions taken by a user (e.g., a professional in some instances); however, it should be understood that they can also include the instruction of those actions by another party. Thus, actions such as “moving a handpiece” or “delivering a fluid” include “instructing moving a handpiece” and “instructing delivering a fluid.” The ranges disclosed herein also encompass any and all overlap, sub-ranges, and combinations thereof. Language such as “up to,” “at least,” “greater than,” “less than,” “between,” and the like includes the number recited. Numbers proceeded by a term such as “about” or “approximately” include the recited numbers. For example, “about 10 mm” includes “10 mm.” Terms or phrases preceded by a term such as “substantially” include the recited term or phrase. For example, “substantially parallel” includes “parallel.”

1-52. (canceled)

53. A tip configured to be secured to a distal end of a handpiece of a skin treatment system, the tip comprising:
a main body portion having a distal end and a proximal end, the proximal end being configured to secure to the handpiece;
a peripheral lip along the distal end, the peripheral lip defining an interior area;
a rollerball positioned within the interior area, the rollerball secured to the tip and configured to rotate when the rollerball is moved relative to a skin surface; and
at least one vacuum port terminating within the interior area and adjacent the rollerball, the at least one vacuum being configured to generate a suction or vacuum along the tip to remove spent fluids and other debris;
wherein the at least one vacuum port is in fluid communication with a vacuum passage or conduit of the tip, the vacuum passage or conduit configured to hydraulically couple to a vacuum or suction source when the tip is secured to the handpiece.

54. The tip of claim 53, wherein the rollerball extends past the peripheral lip or is proud relative to the peripheral lip.

55. The tip of claim 53, wherein the interior area comprises an oval or egg shape.

56. The tip of claim 55, wherein the rollerball is located along a larger diameter portion of the interior area and the at least one vacuum port is located along a smaller diameter portion of the interior area.

57. (canceled)

58. The tip of claim 53, wherein a plane defined by a distal end of the peripheral lip is angled relative to a longitudinal axis of the tip and an axis orthogonal to the longitudinal axis.

59. The tip of claim 53, wherein the peripheral lip is configured to abrade skin tissue when the tip contacts skin tissue and is moved relative to skin tissue.

60. (canceled)

61. A skin treatment system comprising:
a console comprising a manifold assembly, the manifold assembly including at least one fluid connector to be placed in fluid communication with at least one treatment fluid container;
a handpiece configured to hydraulically couple to the manifold assembly, wherein the handpiece is hydraulically coupled to the manifold assembly using at least one fluid conduit;
a processor configured to regulate at least one aspect of an operation of the skin treatment system; and
a user input device configured to receive at least one input or command from a user;
wherein a vacuum source is configured to operatively couple to the system and selectively create suction along the handpiece; and
wherein the user input device comprises a gesture control system.

62. The system of claim 61,
wherein the gesture control system comprises at least two gesture sensors;
wherein the gesture sensors are positioned on, along or near the console; and
wherein the gesture control system is configured to modify a suction created along the handpiece by the vacuum source.

63. The system of claim 61, wherein the gesture control system comprises at least two gesture sensors.

64. The system of claim 61, wherein the gesture control system comprises at least four gesture sensors.

65. The system of claim 63, wherein the gesture sensors are positioned on, along or near the console.

66. The system of claim 65, wherein the gesture sensors are positioned on, along or near at least one of the a top, a left side and a right side of the console.

67. The system of claim 63, wherein the gesture sensors comprise a camera or visual sensor.

68. The system of claim 63, wherein the gesture control system is configured to modify a suction created along the handpiece by the vacuum source.

69. A skin treatment system comprising:
a console comprising a vacuum source;
a handpiece configured to hydraulically couple to the vacuum source, wherein a suction is created along a distal end of the handpiece when the vacuum source is activated and the handpiece is coupled to the console;
wherein the handpiece is configured to receive a container, the container configured to store a treatment material, wherein the treatment material is configured to be delivered to the distal end of the handpiece during use;
a processor configured to regulate at least one aspect of an operation of the skin treatment system; and
an identification tag reader configured to automatically detect at least one identification tag of the container when the container has been positioned within at least one designated recess or other receiving area.

70. The system of claim 69, wherein the at least one designated recess or other received area is located along a tray of the console.

71. The system of claim 69, wherein the identification tag reader comprises a RFID reader.
